(19) United States
(12) Patent Application Publication
(10) Pub. No.: US 2025/0204865 A1
(43) Pub. Date: Jun. 26, 2025

(54) METHODS AND SYSTEMS FOR ESTIMATING BLOOD ANALYTES FROM SPECTROMETER SIGNALS

(71) Applicant: JRE Star Investment Holdings, LLC, Farmington, UT (US)

(72) Inventors: Tyler J. Jarvis, Provo, UT (US); McKayla Davis, Layton, UT (US); Bryce A. Lunceford, Mapleton, UT (US); William R. Terry, Cedar Hills, UT (US); Lydia F. Tollman, Denver, CO (US)

(21) Appl. No.: 18/991,182

(22) Filed: Dec. 20, 2024

Related U.S. Application Data
(60) Provisional application No. 63/612,791, filed on Dec. 20, 2023.

(51) Int. Cl. A61B 5/00; A61B 5/024; A61B 5/145; A61B 5/1455

(52) U.S. Cl. CPC A61B 5/7235; A61B 5/024; A61B 5/14532; A61B 5/14551

(57) ABSTRACT

Methods and systems for estimating blood analyte conditions. In some methods, signal data may be received from a non-invasive blood monitor. The signal data may be used to train a model, which model may use a feature comprising at least two distinct electromagnetic radiation wavelengths, such as a Beer-Lambert inversion model. Following model training, signal data may be received from a non-invasive blood monitor using the at least two distinct electromagnetic radiation wavelengths. The trained model may then be used to estimate a blood analyte condition associated with the blood analyte, such as a concentration of the blood analyte.

### CROSS-REFERENCE TO RELATED APPLICATIONS

This application claims the benefit under 35 U.S.C. § 119(e) of U.S. Provisional Patent Application No. 63/612,791, which was filed Dec. 20, 2023, and titled “Beer-Lambert Inversion for Estimating Blood Analytes from Spectrometer Signal,” which is hereby incorporated herein by reference in its entirety.

### SUMMARY

Noninvasive blood monitoring is a decades-old research problem that has proved to be frustratingly elusive. Data generated from a state-of-the-art infrared spectrometer, combined with new techniques to model physiological interactions, may reveal previously inaccessible signals. Disclosed herein are methods, which may be incorporated into various systems, for deriving distributional estimates for photon scattering and absorption behavior, which lead to simple closed-form models that accurately model photon scattering. These analytic models improve reliable physiological signal extraction and continuous noninvasive measurement of biomarkers of interest.

To reliably determine small signals from noisy data, reasonable models of the underlying signal-generating process must be developed. This has proved to be difficult in the case of, for example, glucose measurement, notably because the light-particle interactions present are difficult to model.

Disclosed herein are therefore models, algorithms, methods, and systems that use Beer-Lambert Inversion techniques to train models and/or work in conjunction with trained models to provide better predictions and/or estimates for various blood analyte conditions, such as concentrations of one or more blood analytes, using non-invasive techniques involving emission of a plurality of distinct wavelengths of electromagnetic radiation in certain wavelength ranges and detection of such radiation reflected from various biological features, including blood, blood vessel walls, and tissue.

Techniques for removing and/or extracting certain components from a pulsatile signal used for estimating certain conditions, such as blood analyte conditions, are also disclosed. For example, because the DC offset component of a non-invasive electromagnetic radiation based sensor system typically contains data that may not be useful, and may make it more difficult to obtain useful information from the data, techniques disclosed herein may be used to extract the DC offset component, which in the case of a non-invasive blood monitoring system may leave only data associated with the pulsatile signal (e.g., the heartbeat being transduced through a blood vessel wall), along with data corresponding to the non-pulsatile blood component of the signal (i.e., electromagnetic waves reflected from the blood itself).

In an example of a method for estimating blood analytes non-invasively according to some implementations, the method may comprise one or more training steps, such as a step of training a model of a relationship between a blood analyte and a non-invasive signal. The model may use a feature comprising at least two distinct electromagnetic radiation wavelengths, which model may comprise a Beer-Lambert inversion model. Signal data may then be received from a non-invasive blood monitor using the at least two distinct electromagnetic radiation wavelengths. The trained model may then be used to estimate a blood analyte condition associated with the blood analyte, such as a concentration of the blood analyte.

In some implementations, the signal may comprise a pulsatile signal. In some such implementations, the pulsatile signal may comprise a heartbeat.

In some implementations, the feature may comprise use of signal values taken at a time of systole and a time of diastole of the heartbeat for two independent wavelengths of the at least two distinct electromagnetic radiation wavelengths. In some such implementations, the signal values may comprise a maximum value and a minimum value taken during a single heartbeat.

In some implementations, the blood analyte may comprise glucose. Alternatively, or additionally, the blood analyte may comprise an ion, hemoglobin, blood enzymes, blood cell counts, lipids, hydration, drugs, hormones, or oxygen.

In some implementations, the model may be configured to estimate a tissue-dependent DC offset component of the signal data. In some such implementations, the method may further comprise processing the signal data to extract the tissue-dependent DC offset component.

Some implementations may comprise evaluating the processed signal data to provide an estimate of a blood analyte condition. In some such implementations, the estimate may be provided using only a frequency component corresponding to a heart rate pulse of a user of the non-invasive blood monitor and a non-pulsatile blood component corresponding to light reflected from within a blood vessel from the signal data.

In some implementations, the Beer-Lambert model may comprise a linear model. Alternatively, the Beer-Lambert model may comprise a non-linear model.

In an example of a method for estimating a blood analyte concentration using data from a non-invasive blood monitor according to other implementations, the method may comprise receiving signal data from a non-invasive blood monitor, wherein the signal data includes data associated with at least two distinct electromagnetic frequencies. A trained model may be used to estimate a blood analyte concentration using the signal data. In some cases, the trained model may comprise a Beer-Lambert inversion model.

In some implementations, the trained model may comprise use of a feature comprising an equation including maximum and minimum values of a signal at two distinct electromagnetic frequencies. In some such implementations, the equation may comprise use of values of the signal at a time of systole and a time of diastole of a heartbeat for the two distinct electromagnetic frequencies. In some cases, the maximum or peak value may correspond to the time of diastole and the minimum or valley value corresponds to the time of systole. However, in alternative implementations, the minimum value may correspond to diastole and the maximum value may correspond to systole, or these values may reverse during data processing.

In some implementations, the signal data may comprise a frequency component corresponding to a heart rate pulse of a user of the non-invasive blood monitor, a non-pulsatile blood component corresponding to light reflected from within a blood vessel, and a tissue-dependent DC offset component.

Some implementations may comprise extracting the tissue-dependent DC offset component from the signal data.

In some implementations, the step of using a trained model to estimate a blood analyte concentration comprises estimating the blood analyte concentration using only the non-pulsatile blood component, or in other cases only using the frequency component and the non-pulsatile blood component.

The features, structures, steps, or characteristics disclosed herein in connection with one embodiment may be combined in any suitable manner in one or more alternative embodiments.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1A is a graph schematically illustrating the constituent components of a signal having a pulsatile portion, a non-pulsatile blood component, and a DC offset/tissue component;

FIG. 1B is a diagram illustrating the various electromagnetic waves that correspond to the signal components illustrated in FIG. 1A;

FIG. 2 is an example of a portion of a design matrix used as part of a Beer-Lambert inversion training algorithm according to some embodiments and implementations;

FIG. 3 is a schematic flowchart illustrating an example of a method for estimating a blood analyte condition using linear Beer-Lambert inversion according to some implementations;

FIG. 4 is a schematic flowchart illustrating an example of a method for estimating a blood analyte condition using non-linear Beer-Lambert inversion according to some implementations; and

FIG. 5 is a flowchart illustrating a more generalized method for estimating a blood analyte condition using Beer-Lambert inversion algorithms according to some implementations.

### DETAILED DESCRIPTION

It will be readily understood that the components of the present disclosure, as generally described and illustrated in the drawings herein, could be arranged and designed in a wide variety of different configurations. Thus, the following more detailed description of the embodiments of the apparatus is not intended to limit the scope of the disclosure but is merely representative of possible embodiments of the disclosure. In some cases, well-known structures, materials, or operations are not shown or described in detail.

As used herein, the term “substantially” refers to the complete or nearly complete extent or degree of an action, characteristic, property, state, structure, item, or result to function as indicated. For example, an object that is “substantially” cylindrical or “substantially” perpendicular would mean that the object/feature is either cylindrical/perpendicular or nearly cylindrical/perpendicular so as to result in the same or nearly the same function. The exact allowable degree of deviation provided by this term may depend on the specific context. The use of “substantially” is equally applicable when used in a negative connotation to refer to the complete or near complete lack of an action, characteristic, property, state, structure, item, or result. For example, structure which is “substantially free of” a bottom would either completely lack a bottom or so nearly completely lack a bottom that the effect would be effectively the same as if it completely lacked a bottom.

Similarly, as used herein, the term “about” is used to provide flexibility to a numerical range endpoint by providing that a given value may be “a little above” or “a little below” the endpoint while still accomplishing the function associated with the range.

The embodiments of the disclosure may be best understood by reference to the drawings, wherein like parts may be designated by like numerals. It will be readily understood that the components of the disclosed embodiments, as generally described and illustrated in the figures herein, could be arranged and designed in a wide variety of different configurations. Thus, the following detailed description of the embodiments of the apparatus and methods of the disclosure is not intended to limit the scope of the disclosure, as claimed, but is merely representative of possible embodiments of the disclosure. In addition, the steps of a method do not necessarily need to be executed in any specific order, or even sequentially, nor need the steps be executed only once, unless otherwise specified. Additional details regarding certain preferred embodiments and implementations will now be described in greater detail with reference to the accompanying drawings.

A classical way to understand light-particle interactions is the Beer-Lambert law, which states that the macroscopic behavior of light when a narrow-beam light source propagates through a diffusive media is governed by the following equation:

$$I = {I}_{0} {e}^{- ε l c}$$

In this equation I is the intensity of detected/transmitted light, I₀ is the initial intensity of the light, ε is the molar absorption coefficient (material dependent), l is the optical path length, and c is the concentration of the absorptive species in the media. Notably, this equation only applies when the path length is constant for all detected paths (or can be approximated to be constant) and the media has a uniform distribution of absorbing media.

Assume we are given a training set consisting of a pulsatile spectrographic signal s(t)=(s_{λ}_{₁}(t), . . . , s_{λ}_{ₙ}(t))∈ [symbol] measured at times t∈{t₁, . . . , t_{T}} with each s_{λ}(t) being the measured value of the spectrometer in wavelength λ at time t. Moreover, assume a(t)=(a₁(t), . . . , a_{K}(t)) is a corresponding sequence of measured or interpolated values of analytes in the blood at each of the times t.

For a given cardiac pulse, the volume of the artery or capillaries through which the light passes will increase during systole and contract during diastole. Let t_{sys} be the time of systole (maximum blood pressure occurring in a given pulse) and t_{dia} be the time of diastole (minimum blood pressure occurring in a given pulse).

Various features may be used for the model, such as features defined by taking raw data, such as raw spectrometer data, at two distinct wavelengths, and then finding the peaks and/or valleys of the signal over a predetermined time period. In some cases, the predetermined time period may correspond to a time period associated with a pulsatile signal, such as a heartbeat. Thus, in some cases, the peaks and/or valleys of the signal may be determined over a single heartbeat. These values may then be combined using a formula, such as the formula provided below.

For any two wavelengths λ₁ and λ₂ measured by the spectrometer, the following computed quantity will be referred to herein as the JMLS feature of the spectrographic signal for wavelengths λ₁ and λ₂ and for the given pulse:

$${J}_{{λ}_{1} , {λ}_{2}} = \frac{log ( {s}_{{λ}_{1}} ( {t}_{sys} ) ) - log ( {s}_{{λ}_{1}} ( {t}_{dia} ) )}{log ( {s}_{{λ}_{2}} ( {t}_{sys} ) ) - log ( {s}_{{λ}_{2}} ( {t}_{dia} ) )} \qquad ( 1 )$$

In this formula, s_{λ}(t) is the raw spectrometer signal measured for wavelength λ over time and t_{sys} is the time of systole (the time of a maxima or minima of the signal; in some cases, the time of the peak of the signal over the heartbeat). t_{dia} is the time of diastole (the time of a maxima or minima of the signal; in some cases, the time of the valley of the signal).

Although any number of wavelengths can be used during this process, in preferred embodiments and implementations, between six and 120 distinct wavelengths may be used simultaneously in modeling to estimate one or more blood analytes. Note that if there are n wavelengths that the spectrometer measures, then there are n(n−1) possible features, such as possible JMLS features. For JMLS features, one wavelength may be chosen as the numerator and one as the denominator for each such feature.

In some cases, one or more modifications to the JMLS feature (and the modification has modifications, etc.) may be implemented. For example, in some cases, a tissue offset value may be calculated, estimated, or otherwise used in the training and/or estimation/prediction steps. In some cases, the tissue offset value may be estimated at zero. Alternatively, the tissue offset may be assumed to be the lowest value of the waveform. As another alternative, the tissue offset may be estimated to be a high percentage of the valley offset, such as 99% of the valley offset.

In still other cases, such modification(s) may involve adding a more particular tissue offset T_{λ} to the JMLS feature, which then provides the following equation, which is explained in greater detail below:

$${J}_{{λ}_{1} , {λ}_{2}} = \frac{log ( {s}_{{λ}_{1}} ( {t}_{sys} ) - {T}_{{λ}_{1}} ) - log ( {s}_{{λ}_{1}} ( {t}_{dia} ) - {T}_{{λ}_{1}} )}{log ( {s}_{{λ}_{2}} ( {t}_{sys} ) - {T}_{{λ}_{2}} ) - log ( {s}_{{λ}_{2}} ( {t}_{dia} ) - {T}_{{λ}_{2}} )} .$$

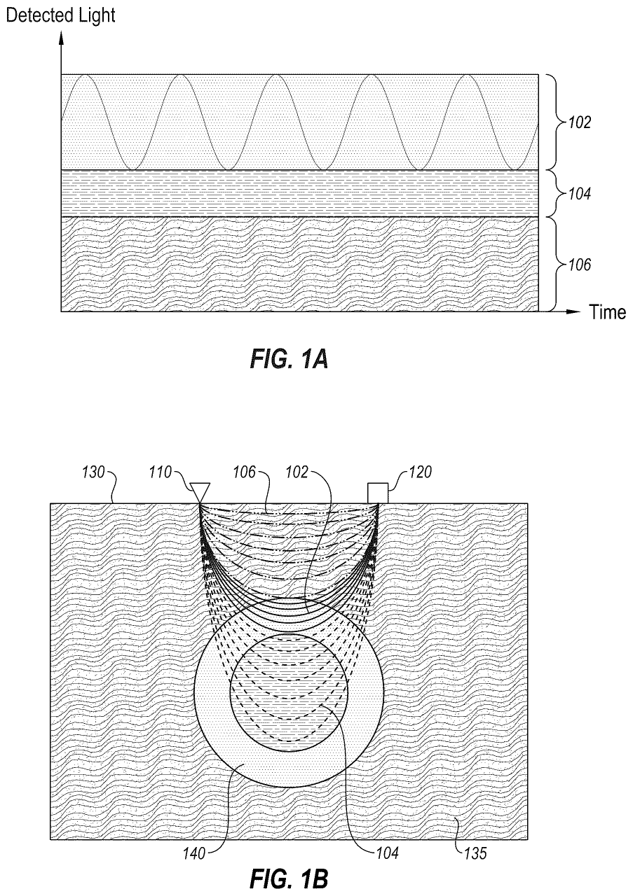

FIGS. 1A and 1B illustrate schematically a system for using electromagnetic radiation from an emitter 110 to process signals and track various physiological conditions. As shown in FIG. 1B, radiation is emitted from emitter 110 and is directed through the skin 130 of a user, through various tissue layers 135, and into a blood vessel 140. The radiation is reflected at various points and received by a sensor 120, which may comprise, for example, a spectrometer. Thus, the signal data referenced throughout this disclosure may be received by a physiological sensor, such as a non-invasive blood monitor. Examples of such monitors can be found in U.S. Pat. No. 11,903,686 titled “Systems, Apparatuses, and Methods for Optimizing a Physiological Measurement Taken From a Subject,” the entire contents of which are hereby incorporated herein by reference in its entirety. Additional details regarding techniques for non-invasive blood monitoring, some of which may be useful in combination with the inventive techniques and/or principles disclosed herein, may be found in U.S. patent application Ser. No. 18/955,868 titled “METHODS AND SYSTEMS FOR ESTIMATING BLOOD ANALYTE CONDITIONS,” which was filed on Nov. 21, 2024, and the entire contents of which are also hereby incorporated by reference.

As shown in FIG. 1B, the sensor 120 may be configured to measure the magnitude/intensity of the received radiation over time. The resulting signal may therefore consist of a pulsatile component 102 (see FIG. 1A), which corresponds to radiation 102 (see FIG. 1B) being reflected from blood vessel 140, as shown in FIG. 1B. Because the blood vessel 140 contracts and expands at a rate corresponding with the heartbeat of the user, this portion of the signal may contain a periodic and/or pulsatile signal, as indicated by the sine wave within pulsatile signal section 102 of the signal.

The signal may also include a non-pulsatile component 104 that results from radiation reflected from within the blood vessel 140. In systems configured to predict, estimate, and/or detect blood analytes, non-pulsatile blood component 104, otherwise referred to herein as the “baseline” component, is likely to contain useful information that may be used to provide a better prediction or estimation of the blood analyte under consideration.

The signal may further comprise a DC offset or tissue component 106. DC offset/tissue component 106 is the result of radiation being reflected from non-blood tissue 135 of the user. Again, if the desired result is to assess blood analyte conditions, this portion of the signal is not likely to contain useful information. Moreover, having this portion of the signal present may detract from the useability of components 102 and 104. This is because prior techniques for processing data with all three components 102/104/106 fail to differentiate between components 104 and 106. For example, it has been typical to simply assume that components 104 and 106 were both resulting from tissue reflections, and therefore did not contain useful information. However, the techniques provided herein allow for not only differentiation of components 104 and 106, but also for extraction of one or both of these components. For example, it may be desirable to extract DC offset/tissue component 106 entirely so that it does not detract from the usefulness of the information in the other two components 102 and 104.

Thus, as described in greater detail below, by using modified features and/or models, such as Beer-Lambert Inversion models incorporating tissue offset components, it is thought that more precise blood analyte estimates may be obtained, at least for certain analytes.

We can now use the Beer-Lambert law to estimate values of a specific blood analyte from the JMLS features, and possibly other, known blood analytes.

For any given wavelength λ, the Beer-Lambert law describes approximately how light entering the body and returning to the spectrometer will be transmitted, assuming that the analytes that change significantly on the time scale of interest (hours or days) are among those that affect light absorption and scattering in the blood significantly are among those measured.

$${s}_{λ} ( t ) \approx {I}_{0 , λ} exp ( - {ℓ}_{b l o} [ {\sum }_{k = 1}^{K} {ε}_{k , λ} {a}_{k} ( t ) ] - {d}_{blo , λ} {ℓ}_{b l o} - {d}_{tis , λ} {ℓ}_{tis} ) , \qquad ( 2 )$$

where the variables are defined as
I_{0,λ} is the initial intensity of light at wavelength A entering the body.
[symbol] _{blo} is the length of the path traveled by the light through the blood.
[symbol] ₜᵢₛ is the length of the path traveled by the light through the surrounding tissue. [symbol]
∈_{k,λ} is the extinction coefficient of light at wavelength A due to the kth blood analyte.
d_{blo,λ} is a constant representing the sum of all the terms of the form ε_{j,λ}aⱼ corresponding to unmeasured analytes in the blood,
d_{tis,λ} is a constant representing the sum of all the terms of the form ε_{j,λ}aⱼ corresponding to unmeasured analytes in the surrounding tissue.

For a given cardiac pulse, the volume of the artery or capillaries through which the light passes will increase during systole and contract during diastole.

Let [symbol] _{sys} be the length of the path traveled by the light through the blood at systole (maximum blood pressure occurring in a given pulse) and

Let [symbol] _{dia} be the length of the path traveled by the light through the blood at diastole (minimum blood pressure occurring in a given pulse).

It is an oversimplification to assume that the various path lengths [symbol] _{*} are the same for all wavelengths or even that they are well-defined for a given wavelength. Indeed, for any wavelength, different photons take different paths from the entry point of body to the spectrometer, and even the expected path in the distribution of all paths has a different length for each wavelength, due to different scattering properties of light at different wavelengths. But we can expect that these should be sufficiently similar for this method to be useful.

For each pulse, we may assume that the blood and tissue analytes do not change significantly in the course of the pulse (or, more precisely, between t_{sys} to t_{dia}). For each wavelength λ and each pulse, in some cases, we can take the difference of (natural) logs of the measured intensities of the signal s_{λ}(t_{sys}) at systole and s_{λ}t_{dia}) at diastole, which causes the terms corresponding to I_{0,λ} and the effect of tissue −d_{tis,λ} [symbol] ₜᵢₛ to cancel out, leading to the following simplification:

$$log ( {s}_{λ} ( {t}_{sys} ) ) - log ( {s}_{λ} ( {t}_{dia} ) ) = - {ℓ}_{sys} ( {\sum }_{k = 1}^{K} {ε}_{k , λ} {a}_{k} ( t ) ) - {d}_{blo , λ} {ℓ}_{sys} - - + {ℓ}_{dia} ( {\sum }_{k = 1}^{K} {ε}_{k , λ} {a}_{k} ( t ) ) + {d}_{dia , λ} {ℓ}_{dia} + . = ( {ℓ}_{dia} - {ℓ}_{sys} ) [ ( {\sum }_{k = 1}^{K} {ε}_{k , λ} {a}_{k} ( t ) ) - {d}_{blo , λ} ] . \qquad ( 3 )$$

The JMLS feature is a ratio of expression (3) above for two different wavelengths λ₁ and λ₂. Assuming that [symbol] _{*} is roughly the same for the two different wavelengths, the difference [symbol] _{sys}− [symbol] _{dia} of path lengths cancels, as indicated below:

$$\begin{matrix}{J}_{{λ}_{1} , {λ}_{2}} = \frac{log ( {s}_{{λ}_{1}} ( {t}_{sys} ) ) - log ( {s}_{{λ}_{1}} ( {t}_{dia} ) )}{log ( {s}_{{λ}_{2}} ( {t}_{sys} ) ) - log ( {s}_{{λ}_{2}} ( {t}_{dia} ) )} \\ = \frac{[ ( {\sum }_{k = 1}^{K} {ε}_{k , {λ}_{1}} {a}_{k} ) - {d}_{blo , {λ}_{1}} ]}{[ ( {\sum }_{k = 1}^{K} {ε}_{k , {λ}_{2}} {a}_{k} ) - {d}_{blo , {λ}_{2}} ]} \\ = \frac{( {\sum }_{k = 1}^{K} {ε}_{k , {λ}_{1}} {a}_{k} ) - {d}_{blo , {λ}_{1}}}{( {\sum }_{k = 1}^{K} {ε}_{k , {λ}_{2}} {a}_{k} ) - {d}_{blo , {λ}_{2}}} .\end{matrix} \qquad ( 4 )$$

Here the values aₖ are locally constant:

$${a}_{k} = {a}_{k} ( {t}_{s y s} ) = {a}_{k} ( {t}_{d i a} )$$

for a given pulse. They are usually essentially constant over a time period of several minutes.

In some preferred embodiments and implementations, a Beer-Lambert inversion may be applied as follows:
Again, in equation (4) above, ε_{k,λ} is the extinction coefficient of light at wavelength λ due to the kth blood analyte, aₖ is the concentration of analyte kin the blood, and d_{blo,λ} is a constant representing the sum of analyte concentrations multiplied by the corresponding extinction coefficients for wavelength λ, for all of the unmeasured analytes in the blood.

First, one may use measured JMLS features and measured analyte concentrations to find estimates of the extinction coefficients \hat{ε}_{k,λ} and/or the blood constants \hat{d}_{blo,λ} of the various constants ε_{k,λ} and d_{blo,λ} for each analyte k and wavelength λ in equation (4) above.

In some cases, this can be done using least squares (optionally with some regularization) after clearing denominators in the equation. In some cases, it may be desirable to include every wavelength of interest either as a numerator or denominator in this system. However, including any wavelength more than once results in a system that is nearly singular (with very small singular values), which generally makes the system less numerically stable.

In some embodiments and implementations, an algorithm may be employed that fixes one value and then uses a least-squares method for estimating the coefficients. In some cases, the first step in this algorithm is to create a “design matrix.” There are many ways to do this, but because we can clear denominators in equation (3), and then subtract the left-hand side, we can write the model in the following equivalent form:

$$0 = ( {\sum }_{k = 1}^{K} {ε}_{k , {λ}_{1}} {a}_{k} ) - {d}_{blo , {λ}_{1}} - {J}_{{λ}_{1} , {λ}_{2}} ( ( {\sum }_{k = 1}^{K} {ε}_{k , {λ}_{2}} {a}_{k} ) - {d}_{blo , {λ}_{2}} )$$

If there are n wavelengths of interest, then there are n(n−1) such equations because there are that many JMLS features. In order to make a linear system, only (n−1) of the equations must be chosen. Which equations are chosen determines how numerically stable the system is. As a baseline, the chosen equations must include all n wavelengths either as a numerator or a denominator.

Any of the n(n−1) equations may be added together to create a new equation and equations crafted in this way will remain mathematically valid. Thus, there are an endless number of schemes that could be used to choose the equations that build the design matrix. Despite there being an endless number of possible schemes that could be used to choose the equations, none of the schemes is substantively different from any of the others. The only difference is numerical stability.

Once a scheme is chosen, the scheme builds (n−1)×(n·#numanalytes) sized blocks of the design matrix. Every peak/valley and/or maxima/minima pair in the spectrometer signal creates a block, and the blocks may be placed (row-wise) consecutively to build the design matrix. Using all of the available data in the spectrometer signal creates a very large (thousands to millions of rows) design matrix.

As an example of how the design matrix may be built in some cases, suppose that there are eight wavelengths used and only one analyte being sought for estimation. One of the simplest schemes that could be chosen to build the design matrix would be to choose all of the equations that have, e.g., λ₅ in the denominator. This would give the following 7 equations:

$$0 = ε0 , {λ}_{0} a 0 - dblo , {λ}_{0} - J {λ}_{0} , {λ}_{5} ε 0 , {λ}_{5} a 0 + J {λ}_{0} , {λ}_{5} dblo , {λ}_{5} \\ 0 = ε0 , {λ}_{1} a 0 - dblo , {λ}_{1} - J {λ}_{1} , {λ}_{5} ε 0 , {λ}_{5} a 0 + J {λ}_{1} , {λ}_{5} dblo , {λ}_{5} \\ 0 = ε0 , {λ}_{2} a 0 - dblo , {λ}_{2} - J {λ}_{2} , {λ}_{5} ε 0 , {λ}_{5} a 0 + J {λ}_{2} , {λ}_{5} dblo , {λ}_{5} \\ 0 = ε0 , {λ}_{3} a 0 - dblo , {λ}_{3} - J {λ}_{3} , {λ}_{5} ε 0 , {λ}_{5} a 0 + J {λ}_{3} , {λ}_{5} dblo , {λ}_{5} \\ 0 = ε0 , {λ}_{4} a 0 - dblo , {λ}_{4} - J {λ}_{4} , {λ}_{5} ε 0 , {λ}_{5} a 0 + J {λ}_{4} , {λ}_{5} dblo , {λ}_{5} \\ 0 = ε0 , {λ}_{6} a 0 - dblo , {λ}_{6} - J {λ}_{6} , {λ}_{5} ε 0 , {λ}_{5} a 0 + J {λ}_{6} , {λ}_{5} dblo , {λ}_{5} \\ 0 = ε0 , {λ}_{7} a 0 - dblo , {λ}_{7} - J {λ}_{7} , {λ}_{5} ε 0 , {λ}_{5} a 0 + J {λ}_{7} , {λ}_{5} dblo , {λ}_{5}$$

These equations can be expressed equivalently as the following matrix equation:

$$[ \begin{matrix}{a}_{0} & 0 & 0 & 0 & 0 & - {J}_{{λ}_{0} , {λ}_{5}} {a}_{0} & 0 & 0 & - 1 & 0 & 0 & 0 & 0 & {J}_{{λ}_{0} , {λ}_{5}} & 0 & 0 \\ 0 & {a}_{0} & 0 & 0 & 0 & - {J}_{{λ}_{1} , {λ}_{5}} {a}_{0} & 0 & 0 & 0 & - 1 & 0 & 0 & 0 & {J}_{{λ}_{1} , {λ}_{5}} & 0 & 0 \\ 0 & 0 & {a}_{0} & 0 & 0 & - {J}_{{λ}_{2} , {λ}_{5}} {a}_{0} & 0 & 0 & 0 & 0 & - 1 & 0 & 0 & {J}_{{λ}_{2} , {λ}_{5}} & 0 & 0 \\ 0 & 0 & 0 & {a}_{0} & 0 & - {J}_{{λ}_{3} , {λ}_{5}} {a}_{0} & 0 & 0 & 0 & 0 & 0 & - 1 & 0 & {J}_{{λ}_{3} , {λ}_{5}} & 0 & 0 \\ 0 & 0 & 0 & 0 & {a}_{0} & - {J}_{{λ}_{4} , {λ}_{5}} {a}_{0} & 0 & 0 & 0 & 0 & 0 & 0 & - 1 & {J}_{{λ}_{4} , {λ}_{5}} & 0 & 0 \\ 0 & 0 & 0 & 0 & 0 & - {J}_{{λ}_{6} , {λ}_{5}} {a}_{0} & {a}_{0} & 0 & 0 & 0 & 0 & 0 & 0 & {J}_{{λ}_{6} , {λ}_{5}} & - 1 & 0 \\ 0 & 0 & 0 & 0 & 0 & - {J}_{{λ}_{7} , {λ}_{5}} {a}_{0} & 0 & {a}_{0} & 0 & 0 & 0 & 0 & 0 & {J}_{{λ}_{7} , {λ}_{5}} & 0 & - 1\end{matrix} ] [ \begin{matrix}{ε}_{0 , {λ}_{0}} \\ {ε}_{0 , {λ}_{1}} \\ {ε}_{0 , {λ}_{2}} \\ {ε}_{0 , {λ}_{3}} \\ {ε}_{0 , {λ}_{4}} \\ {ε}_{0 , {λ}_{5}} \\ {ε}_{0 , {λ}_{6}} \\ {ε}_{0 , {λ}_{7}} \\ {d}_{blo , {λ}_{0}} \\ {d}_{blo , {λ}_{1}} \\ {d}_{blo , {λ}_{2}} \\ {d}_{blo , {λ}_{3}} \\ {d}_{blo , {λ}_{4}} \\ {d}_{blo , {λ}_{5}} \\ {d}_{blo , {λ}_{6}} \\ {d}_{blo , {λ}_{7}}\end{matrix} ] = 0$$

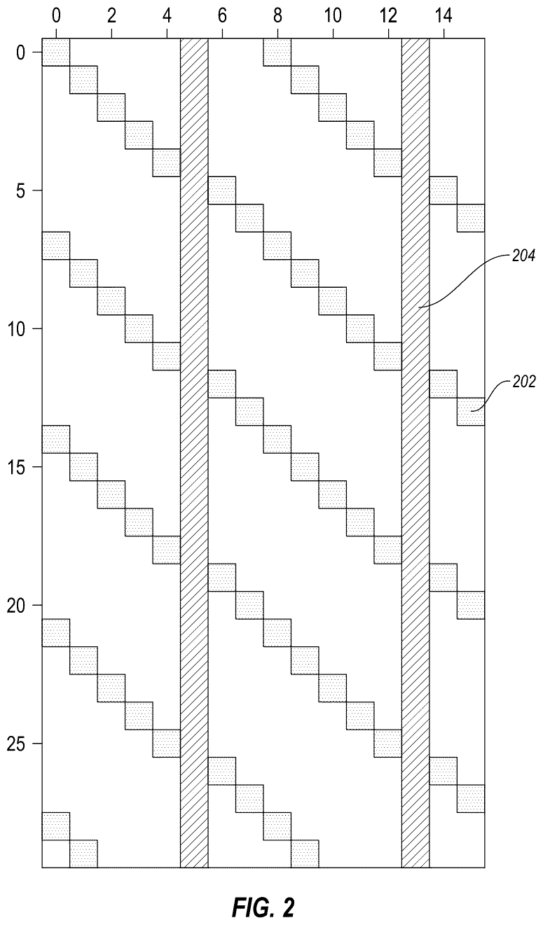

The matrix above is one block of the design matrix according to some implementations. To make the full design matrix, several of these blocks may be stacked, one block for every peak/valley and/or maxima/minima pair. The full design matrix then looks (qualitatively) like the image in FIG. 2.

FIG. 2 illustrates an example of a design matrix according to some implementations. This figure shows the first thirty rows of the matrix to provide a qualitative picture. The full design matrix will likely have thousands to millions of rows. The diagonally extending boxes 202 indicate values that come from the numerator of equation (3), while vertical boxes 204 indicate values from the denominator of this equation.

The system of equations is homogenous, so its solution is the trivial solution of all zeros. This solution is not helpful, so to find a non-trivial solution one can fix one of the unknown values (one of the ε_{k,λ} or d_{blo,λ} values) to 1. Using the example scheme shown, the value chosen is usually d_{blo,λ5}, the d_{blo,λ} value for the wavelength in the denominator.

When fixing the value, the corresponding column from the design matrix may be removed, but a copy of it may be kept for later use. For simplicity, the design matrix with the column removed will be referred to herein as the A matrix, and the removed column the b vector.

Finally, to find the unknown variables, least-squares may be applied to solve the system Ax=−b. The found x vector contains the learned values of the (previously) unknown variables. This completes the first training algorithm.

A second and/or alternative training algorithm may be used in some cases. In some such cases, this algorithm may begin similar to the previous algorithm. First, the design matrix may be built, as described above.

However, instead of fixing one of the values and then solving a system via least-squares, the truncated singular value decomposition of the design matrix may be taken. If A is the design matrix, then, taking the truncated singular value decomposition, A may be written as

A=∪ΣV^{T}

The values of the unknown variables are contained in the last column of the V matrix (the column that corresponds to the smallest singular value).

As still another alternative algorithm, which may be used with, or as an alternative to, any of the algorithms referenced above, one may solve for the Beer-Lambert constants by dropping the assumptions of linear relationships between the parameters and constructing a loss function the global minimum of which is achieved for correct values. Automatic differentiation can then be used to apply gradient descent, L-BFGS (Broyden-Fletcher-Goldfarb-Shanno), or some other gradient-based method to iteratively obtain better values.

The training step may yield Beer-Lambert constants, an additional set of parameters contained in a vector ⁻r, and an estimated analyte vector \hat{ }a. We may then take the learned Beer-Lambert constants from this step and carry them to the prediction step discussed below.

$${R}_{{λ}_{i} , {λ}_{j}} = ( {\sum }_{k = 1}^{K} {\hat{ϵ}}_{k , {λ}_{i}} {\hat{a}}_{k} ) - {\hat{d}}_{blo , {λ}_{i}} - {r}_{{λ}_{i} , {λ}_{j}} [ ( {\sum }_{k = 1}^{K} {\hat{ϵ}}_{k , {λ}_{j}} {\hat{a}}_{k} ) - {\hat{d}}_{blo , {λ}_{j}} ] . \qquad ( 5 )$$

R̅ may be defined as the vector constructed from each pair of {λᵢ,λⱼ}. J̅ may be defined the same way using the JMLS feature. We can then construct a loss function which is minimized when all BLI parameters are correct.

$${arg min}_{\hat{ϵ} , \hat{a} , \hat{d} , \overline{r}} \frac{1}{m} ‖ \overline{J} - \overline{r} ‖ + \frac{μ}{m} ‖ \overline{R} ‖ + {\sum }_{k = 1}^{K} {v}_{k} ( {a}_{k} - {\overline{a}}_{k} ) . \qquad ( 6 )$$

μ and v are hyperparameters set so the contribution of each segment of the loss function is roughly the same order of magnitude.

After training, the learned BLI parameters \hat{ }ϵ and d are treated as fixed. We can then solve a slightly smaller optimization problem to learn analyte values â. After the parameters are learned by way of training optimization, in some cases a secondary linear or nonlinear optimization problem may be used to estimate analyte conditions, such as analyte concentrations.

$${arg min}_{\hat{a} , \overline{r}} \frac{1}{m} ‖ \overline{J} - \overline{r} ‖ + \frac{μ}{m} ‖ \overline{R} ‖ . \qquad ( 7 )$$

After learning the BLI parameters from the training step, as described above in connection with the linear method for performing the prediction to be used following linear system training, we can now solve a related but different linear system to extract analyte prediction values:

$${d}_{blo , {λ}_{j}} {J}_{{λ}_{i} , {λ}_{j}} - {d}_{blo , {λ}_{i}} = [ {\sum }_{k = 1}^{K} {ε}_{k , {λ}_{i}} {a}_{k} ] {J}_{{λ}_{i} , {λ}_{j}}$$

This can be rewritten as a linear system h=Ha⁻ where each row corresponds to the equation above for a specific wavelength pair and ⁻a is the analyte predictions in vector form.

In some implementations and embodiments, use of wavelengths in or around the near infrared (NIR) may be useful. In some cases, visible wavelengths may be used. Thus, for certain blood analytes, such as glucose for example, wavelengths in the range of between about 450 nm and about 3000 nm may be used. In some cases, wavelengths of between about 1000 nm and about 3000 nm may be used. In some such cases, the wavelength range may be between about 2000 nm and about 3000 nm. For certain applications, an even narrower range of wavelengths may be used, such as a range between about 2500 nm to about 3000 nm.

Note that the resulting system of equations in linear modeling is non-homogeneous, so the solution(s), such as one or more analyte concentrations, may be unique.

Second, for a working data set with spectrographic measurements but with some missing analyte measurements, in some cases we can use the estimated constants and the equation (4) above to construct a linear system to solve (again, by least squares, possibly with a regularizer, for example) certain unmeasured analytes as a function of the JMLS values and possibly other measured analytes.

Third, if the predicted values of the analytes change on a time scale much faster than expected for that analyte, in some cases, the results may be smoothed, such as, for example, by use of a Kalman filter with the unknown analyte levels as the hidden states and the identity as the state-transition function, reflecting the fact that the analytes are essentially constant on the time frame of a single pulse.

As mentioned above, in some preferred embodiments and implementations, the Beer-Lamber Inversion may be applied with a tissue offset. As previously mentioned, the Beer-Lambert law is a simplified model of how light passes through tissue and blood in the body. For some applications, a better model may be one that includes separate terms for light passing through the tissue and light passing through the blood, so that equation (2) can be replaced with:

$${s}_{λ} ( t ) \approx {I}_{0 , λ} exp ( - {ℓ}_{tiss} {\overline{ε}}_{λ} \overline{{a}_{tiss}} ) + {I}_{0 , λ} exp ( - {ℓ}_{b l o} [ {\sum }_{k = 1}^{K} {ε}_{k , λ} {a}_{k} ( t ) ] - {d}_{b l o , λ} {ℓ}_{b l o} ) , \qquad ( 8 )$$

where [symbol] ₜᵢₛₛ represents the mean path length through the tissue, ε̅_{λ} is an average extinction coefficient for all analytes in the tissue, and a̅ₜ̅ᵢ̅ₛ̅ₛ̅ an appropriately-weighted average concentration amount for all analytes in the tissue. Since these often cannot be measured reliably, in some cases we can combine them into a single constant T_{λ}, which we can consider the tissue offset and then we can rewrite equation (8) as:

$${s}_{λ} ( t ) \approx {T}_{λ} + {I}_{0 , λ} exp ( - {ℓ}_{b l o} [ {\sum }_{k = 1}^{K} {ε}_{k , λ} {a}_{k} ( t ) ] - {d}_{blo , λ} {ℓ}_{b l o} ) , \qquad ( 9 )$$

Under this model, we can replace s_{λ}(t_{sys} and s_{λ}(t_{dia}) with s_{λ}(t_{sys}−T_{λ} and s_{λ}t_{dia})−T_{λ} in equation (1) to get the following, modified JMLS feature:

(10)

$${J}_{{λ}_{1} , {λ}_{2}}^{T} = \frac{log ( {s}_{{λ}_{1}} ( {t}_{sys} ) - {T}_{λ} ) - log ( {s}_{{λ}_{1}} ( {t}_{dia} ) - {T}_{λ} )}{log ( {s}_{{λ}_{2}} ( {t}_{sys} ) - {T}_{λ} ) - log ( {s}_{{λ}_{2}} ( {t}_{dia} ) - {T}_{λ} )} .$$

The corresponding adjustment to equation (3) gives:

$$log ( {s}_{λ} ( {t}_{s y s} ) - {T}_{λ} ) - log ( {s}_{λ} ( {t}_{d i a} ) - {T}_{λ} ) = ( {ℓ}_{d i a} - {ℓ}_{s y s} ) [ ( {\sum }_{k = 1}^{K} {ε}_{k , λ} {a}_{k} ( t ) ) - {d}_{b l o , λ} ] . \qquad ( 11 )$$

And the adjusted version of equation (4) then becomes:

$${J}_{{λ}_{1} , {λ}_{2}}^{T} = \frac{( {\sum }_{k = 1}^{K} {ε}_{k , {λ}_{1}} {a}_{k} ) - {{d}_{b l o , λ}}_{1}}{( {\sum }_{k = 1}^{K} {ε}_{k , {λ}_{2}} {a}_{k} ) - {{d}_{b l o , λ}}_{2}} . \qquad ( 12 )$$

If T_{λ} is known for each A, then the modified JMLS features J_{λ}_{₁}_{,λ}_{₂} ^{T} can be used in the Beer-Lambert inversion algorithm in the same way as the original (unmodified) JMLS features are.

However, T_{λ} is not known. To estimate it, several methods can be used. For example, we can use a loss function [symbol] (T) depending on all the T_{λ}s. The loss function measures the failure of the modified Beer-Lambert inversion (for a given set of Ts) to give the correct analytes. This could be either the mean-squared error loss or the negative log likelihood computed from the output of the Kalman filter or some other differentiable loss function. Because [symbol] (T) is (automatically) differentiable, we can use a standard optimization algorithm (gradient descent, stochastic gradient descent, L-BFGS) to find the estimated optimal values \hat{T}_{λ} that give the best results on the training set. The trained model consists of all the TAs as well as the estimated parameters \hat{ε}_{k,λ} and \hat{d}_{blo,λ} for each k and λ determined for that specific set of offset values \hat{T}_{λ}.

The rest of the algorithm may then proceed as before. For a working data set with spectrographic measurements but with some missing analyte measurements, the estimated constants and the equation (9) may be used to construct a linear system to solve (again, by least squares, possibly with a regularizer, for example) certain unmeasured analytes as a function of the adjusted JMLS values and possibly other measured analytes. If the predicted values of the analytes change on a time scale much faster than expected for that analyte, the results may be smoothed, such as using a Kalman filter.

In some embodiments and implementations, nonlinear optimization of the tissue offset may be used.

Consider the estimate of tissue offset in a certain wavelength T\hat{ }^{λ}. With the assumption of constant tissue offset (i.e., the tissue offset for a specific wavelength does not drift over time), then the following relationship holds only if the tissue offset values are correct:

$$\frac{log ( {s}_{{λ}_{1}} ( {t}_{sys} ) - {\hat{T}}_{{λ}_{1}} ) - log ( {s}_{{λ}_{1}} ( {t}_{dia} ) - {\hat{T}}_{{λ}_{1}} )}{log ( {s}_{{λ}_{2}} ( {t}_{sys} ) - {\hat{T}}_{{λ}_{2}} ) - log ( {s}_{{λ}_{2}} ( {t}_{dia} ) - {\hat{T}}_{{λ}_{2}} )} = \frac{( {\sum }_{k = 1}^{K} {ε}_{k , {λ}_{1}} {a}_{k} ) - {{d}_{b l o , λ}}_{1}}{( {\sum }_{k = 1}^{K} {ε}_{k , {λ}_{2}} {a}_{k} ) - {{d}_{b l o , λ}}_{2}} ,$$

which we will assign to the term δ(T_{λi}, T_{λj}), which will evaluate to value 0 for correct tissue offsets. We can thus formulate an optimization problem which can be solved with gradient-based methods.

$${arg min}_{\hat{T}} \frac{1}{N} {\sum }_{i , j} δ {( {T}_{{λ}_{i}} , {T}_{{λ}_{j}} )}^{2} ,$$

where N is the number of wavelength pairs.

An assumption of the tissue offset model discussed above is that the offset in each wavelength takes a constant value without regard to the absolute signal strength or variations that happen over time. In some cases, a potentially more useful model may have the DC offset as a percentage of the total radiation that reaches the detector.

In many or most cases involving non-invasive blood monitoring, it is expected that most of the radiation emitted by the emitter does not reach the detector/spectrometer, and/or that most of the radiation that does reach the detector does not interact with the artery/vessel wall in a meaningful way. Thus, a potentially useful model for tissue offset would incorporate a value of around 95-99 percent of the total observed signal at diastole:

T_{λ}=β_{λ}s_{λ}(t_{dia}),β_{λ}∈[0.95,1).

The specific value of β_{λ} could be considered a patient, device, or otherwise specific hyperparameter that needs to be explicitly set before BLI can be performed. The modified JMLS feature then becomes:

$${J}_{{λ}_{1} , {λ}_{2}}^{T} = \frac{log ( {s}_{{λ}_{1}} ( {t}_{sys} ) - {β}_{{λ}_{1}} {s}_{{λ}_{1}} ( {t}_{dia} ) ) - log ( {s}_{{λ}_{1}} ( {t}_{dia} ) - {β}_{{λ}_{1}} {s}_{{λ}_{1}} ( {t}_{dia} ) )}{log ( {s}_{{λ}_{2}} ( {t}_{sys} ) - {β}_{{λ}_{2}} {s}_{{λ}_{2}} ( {t}_{dia} ) ) - log ( {s}_{{λ}_{2}} ( {t}_{dia} ) - {β}_{{λ}_{2}} {s}_{{λ}_{2}} ( {t}_{dia} ) )} .$$

In some embodiments and implementations, offset may be incorporated into nonlinear optimization. For example, one can add the multiplicative offset parameters to the nonlinear optimization problem by modifying the JMLS vector term to include offset term T=βs(t_{dia}).

$${arg min}_{ϵ , \hat{a} , d , \overline{r} , β} \frac{1}{m} ‖ {\overline{J}}^{T} - \overline{r} ‖ + \frac{μ}{m} ‖ \overline{R} ‖ + {\sum }_{k = 1}^{k} {v}_{k} ( {a}_{k} - {\hat{a}}_{k} ) .$$

The same may be done to the prediction problem:

$${arg min}_{\hat{a} , \overline{r}} \frac{1}{m} ‖ {\overline{J}}^{T} - \overline{r} ‖ + \frac{μ}{m} ‖ \overline{R} ‖ .$$

In practice, however, it is possible to learn negative analyte values for âₖ since we have no domain restrictions on it, nor do we have a domain restriction on T, so it could take a value greater than one.

To combat this, we may, in some cases, use a bijective function to map a learned unconstrained parameter to the constrained domain we want.

For example, for \hat{ }a, we may use the exponential function:

$$g ( x ) = exp ( x ) , {g}^{{}_{- 1}} ( x ) = log ( x ) .$$

For β, we may use the sigmoid function:

$$h ( x ) = \frac{1}{1 + exp ( - x )} , {h}^{- 1} ( x ) = log ( \frac{x}{1 - x} ) .$$

The training optimization problem is now:

$${arg min}_{ϵ , {\hat{a}}^{*} , d , \overline{r} , {β}^{*}} \frac{1}{m} ‖ {\overline{J}}^{T} - \overline{r} ‖ + \frac{μ}{m} ‖ \overline{R} ‖ + {\sum }_{k = 1}^{k} {v}_{k} ( {a}_{k} - g ( {\hat{a}}_{k}^{*} ) ) .$$

with T=h(β*)s(t_{dia}) in J̅^{T}. These modifications may also be made to the prediction optimization problem:

$${arg min}_{{\hat{a}}^{*} , \overline{r}} \frac{1}{m} ‖ {\overline{J}}^{T} - \overline{r} ‖ + \frac{μ}{m} ‖ \overline{R} ‖ .$$

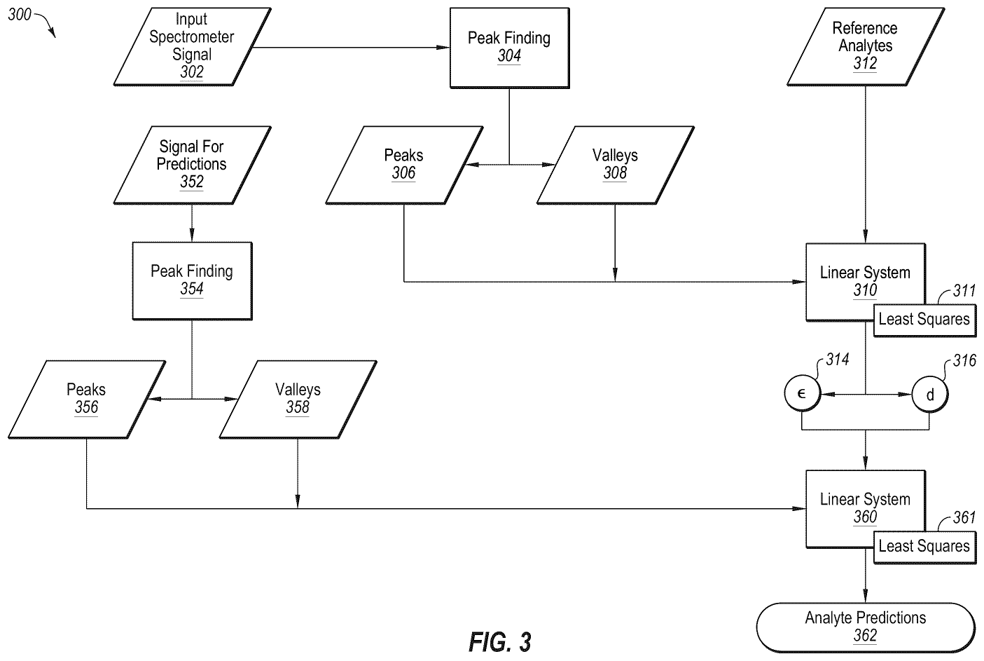

FIG. 3 is a flowchart illustrating an example of a method 300 for processing signal data to estimate, calculate, and/or predict a condition, such as a blood analyte condition, according to some implementations. Examples of possible blood analytes that may be detected, estimated, and/or predicted using method 300 include glucose, ions, hemoglobin, blood enzymes, blood cell counts, lipids, hydration, drugs, hormones, and oxygen. Conditions detected, estimated, and/or predicted for one or more such analytes include, for example, concentrations and/or concentration trends over time.

Method 300 involves use of linear Beer-Lambert inversion. Method 300 comprises a training branch, which begins at step 302, and an estimation and/or prediction branch, which begins at step 352, either branch of which may be separated from the other in some cases and considered an independent invention. The training branch may be generalized or personalized. For example, in some implementations, the training branch may be trained for general applicability among a population, or sub-population, of users. Alternatively, for more precise results, in some cases the training branch may be trained for a particular user, which may provide better results for certain analytes.

At step 302, signal data may be received for training and/or calibration of a model. In some cases, such data may be received from a spectrometer or other detector/sensor, preferably non-invasively. In some cases, the signal data may be multi-channel signal data. For example, each channel of such a multi-channel signal may correspond with a distinct wavelength, wavelength range, and/or sensor. This may provide for enhanced detection capabilities by taking advantage of different absorption characteristics of glucose or other blood analytes at various wavelengths, typically within the near-infrared (NIR) spectrum, as mentioned above. As also previously mentioned, the signal data may be received from a blood monitoring device and/or system, which may comprise a wearable device, such as a wrist monitor, that may be configured with the aforementioned emitter(s) and sensor(s).

Although use of a single channel of signal data may be feasible for some uses, a multi-channel signal may allow for capturing the signal at different settings.

At step 304, the data may be processed to find a maxima and/or minima of the signal within a predetermined time period, such as within a single pulse and/or heartbeat of a pulsatile signal and/or heartbeat signal. In some cases, step 304 may comprise determining a peak of the signal, such as a peak of the signal at a time of diastole, for example, as discussed in detail above. However, in some cases, the minima and maxima may be reversed without losing functionality. Moreover, in some cases, the minima and maxima, such as the peak and valley of the signal, may reverse in the middle of a data processing routine.

Steps 306 and 308 may comprise finding and/or processing a formula involving both the maxima and minima of the signal over the predetermined time period, such as over a single beat of the heart, as mentioned. Thus, in some cases, steps 306 and 308 may comprise finding the peaks and valleys of the signal over a single beat of the heart and combining them using one or more features, such as the JMLS feature described in detail above.

Processed data from steps 306 and 308 may then be used at step 310 to construct a linear system. Reference analytes 312 may be used in some implementations during training in order to calibrate and/or improve the system. For example, in some cases, precise measurements of the blood analytes, such as blood analyte concentrations, may be made, in some cases using invasive methods involving blood draws. Such data may be used to improve the accuracy of the training and, ultimately, the accuracy of the estimations and/or predictions made by the non-invasive branch of the method 300.

As indicated at 311, in some cases, this linear system may be solved using a least-squares method, which may, as described above, allow for finding unknown values and/or parameters, such as extinction coefficients e and/or blood constants d in a Beer-Lambert model, as indicated at 314 and 316 in method 300.

Following and/or concurrently with the training steps of method 300, the prediction/estimation portion of method 300 may also proceed. Thus, at step 352, signal data may be received that will be used in the trained model to estimate and/or predict one or more conditions of one or more blood analytes, such as blood glucose concentrations in some preferred implementations.

As with the training steps, such data may be received from a spectrometer or other detector/sensor, preferably non-invasively. In some cases, the signal data may be multi-channel signal data. For example, each channel of such a multi-channel signal may correspond with a distinct wavelength, wavelength range, and/or sensor. This may provide for enhanced detection capabilities by taking advantage of different absorption characteristics of glucose or other blood analytes at various wavelengths, typically within the near-infrared (NIR) spectrum, as mentioned above. As also previously mentioned, the signal data may be received from a blood monitoring device and/or system, which may comprise a wearable device, such as a wrist monitor, that may be configured with the aforementioned emitter(s) and sensor(s).

In some implementations, wavelengths in or around the near infrared (NIR) may be useful. In some cases, some visible wavelengths may be used as well. Thus, for certain blood analytes, such as glucose for example, wavelengths in the range of between about 450 nm and about 3000 nm may be used. In some cases, wavelengths of between about 1000 nm and about 3000 nm may be used. In some such cases, the wavelength range may be between about 2000 nm and about 3000 nm. For certain applications, an even narrower range of wavelengths may be used, such as a range between about 2500 nm to about 3000 nm.

At step 354, the data may be processed to find a maxima and/or minima of the signal within a predetermined time period, such as within a single pulse and/or heartbeat of a pulsatile signal and/or heartbeat signal. In some cases, step 354 may comprise determining a peak of the signal, such as a peak of the signal at a time of diastole, for example, as discussed in detail above.

Steps 356 and 358 may comprise finding and/or processing a formula involving both the maxima and minima of the signal over the predetermined time period, such as over a single beat of the heart, as mentioned. Thus, in some cases, steps 356 and 358 may comprise finding the peaks and valleys of the signal over a single beat of the heart and combining them in a linear system, as indicated at 360, using the learned coefficients, and/or parameters from the training branch of method 300, some of which may be considered constants in one or more algorithms, such as a Beer-Lambert Inversion prediction algorithm discussed above.

As indicated by the optional feature shown at 361, in some cases a least-squares method, in some such cases along with a regularizer, may be used to solve this equation.

The resulting data from these steps may then be used at step 362 to provide predictions and/or estimations about one or more blood analytes, including any of the blood analytes mentioned herein.

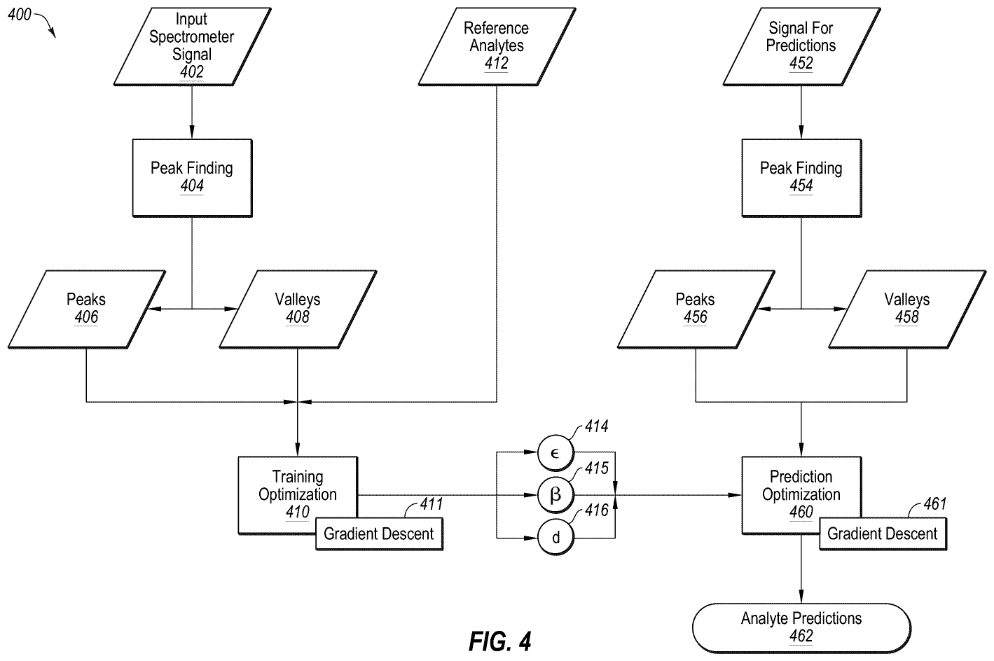

Another method 400 according to some implementations is illustrated in the flowchart of FIG. 4. As with method 300, method 400 comprises a training branch and an estimation/prediction branch. Again, either branch may be performed independently of the other in some implementations or may be combined together in other cases. Method 400 may be considered a method for processing signal data to estimate, calculate, and/or predict a condition, such as a blood analyte condition, according to some implementations, including, for example, any of the blood analytes previously mentioned. Unlike method 300, however, method 400 comprises a non-linear Beer-Lambert Inversion methodology.

As previously mentioned, the training branch of method 400, which begins at step 402, may be generalized or personalized. For example, the training branch may be trained for general applicability among a population, or sub-population, of users. Alternatively, for more precise results, in some cases the training branch may be trained for a particular user, which may provide better results for certain analytes.

At step 402, signal data may be received for training and/or calibration of a model. In some cases, such data may be received from a spectrometer or other detector/sensor, preferably non-invasively. In some cases, the signal data may be multi-channel signal data. For example, each channel of such a multi-channel signal may correspond with a distinct wavelength, wavelength range, and/or sensor. Again, the signal data may in some cases be received from a blood monitoring device and/or system, which may comprise a wearable device, such as a wrist monitor, that may be configured with the aforementioned emitter(s) and sensor(s).

At step 404, the data may be processed to find a maxima and/or minima of the signal within a predetermined time period, such as within a single pulse and/or heartbeat of a pulsatile signal and/or heartbeat signal. In some cases, step 404 may comprise determining a peak of the signal, such as a peak of the signal at a time of diastole, for example, as discussed in detail above. Again, however, in some implementations, the peak/maxima/maximum and valley/minima/minimum may be reversed, including reversals during a data processing routine.

Steps 406 and 408 may comprise finding and/or processing a formula involving both the maxima and minima of the signal over the predetermined time period, such as over a single beat of the heart, as mentioned. Thus, in some cases, steps 406 and 408 may comprise finding the peaks and valleys of the signal over a single beat of the heart and combining them using one or more features, such as the JMLS feature described in detail herein.

Processed data from steps 406 and 408 may then be used at step 410 to optimize the training, which in some cases may consist of constructing a non-linear system and/or non-linear optimization using any of the techniques, methods, and/or algorithms disclosed herein. Reference analytes 412 may be used in some implementations during training in order to calibrate and/or improve the system. For example, in some cases, precise measurements of the blood analytes, such as blood analyte concentrations, may be made, in some cases using invasive methods involving blood draws. Such data may be used to improve the accuracy of the training and, ultimately, the accuracy of the estimations and/or predictions made by the non-invasive branch of the method 400.

As indicated at 411, in some cases, this non-linear system may be solved using a gradient-based method, which may, as described above, allow for finding unknown values and/or parameters, such as such as extinction coefficients e, offset parameters p, and/or blood constants d in a non-linear Beer-Lambert model, as indicated at 414, 415, and 416 in method 400.

Following and/or concurrently with the training steps of method 400, the prediction/estimation portion of method 400 may also proceed. Thus, at step 452, signal data may be received that will be used in the trained model to estimate and/or predict one or more conditions of one or more blood analytes, such as blood glucose concentrations in some preferred implementations.

As with the training steps, such data may be received from a spectrometer or other detector/sensor, preferably non-invasively. In some cases, the signal data may be multi-channel signal data. For example, each channel of such a multi-channel signal may correspond with a distinct wavelength, wavelength range, and/or sensor. This may provide for enhanced detection capabilities by taking advantage of different absorption characteristics of glucose or other blood analytes at various wavelengths, typically within the near-infrared (NIR) spectrum, as mentioned above. As also previously mentioned, the signal data may be received from a blood monitoring device and/or system, which may comprise a wearable device, such as a wrist monitor, that may be configured with the aforementioned emitter(s) and sensor(s).

Again, wavelengths in or around the near infrared (NIR) may be useful. In some cases, some visible wavelengths may be used as well. Thus, for certain blood analytes, such as glucose for example, wavelengths in the range of between about 450 nm and about 3000 nm may be used. In some cases, wavelengths of between about 1000 nm and about 3000 nm may be used. In some such cases, the wavelength range may be between about 2000 nm and about 3000 nm. For certain applications, an even narrower range of wavelengths may be used, such as a range between about 2500 nm to about 3000 nm.

At step 454, the data may be processed to find a maxima and/or minima of the signal within a predetermined time period, such as within a single pulse and/or heartbeat of a pulsatile signal and/or heartbeat signal. In some cases, step 454 may comprise determining a peak of the signal, such as a peak of the signal at a time of diastole, for example, as discussed in detail above.

Steps 456 and 458 may comprise finding and/or processing a formula involving both the maxima and minima of the signal over the predetermined time period, such as over a single beat of the heart, as mentioned. Thus, in some cases, steps 456 and 458 may comprise finding the peaks and valleys of the signal over a single beat of the heart and combining them in a non-linear prediction and/or optimization model, as indicated at 460, using the learned coefficients, constants, and/or parameters from the training branch of method 400.

As indicated by the optional feature shown at 461, in some cases, a gradient-based method, such as L-BFGS, may be used to apply gradient descent.

The resulting data from these steps may then be used at step 462 to provide predictions and/or estimations about one or more blood analytes, including any of the blood analytes mentioned herein.

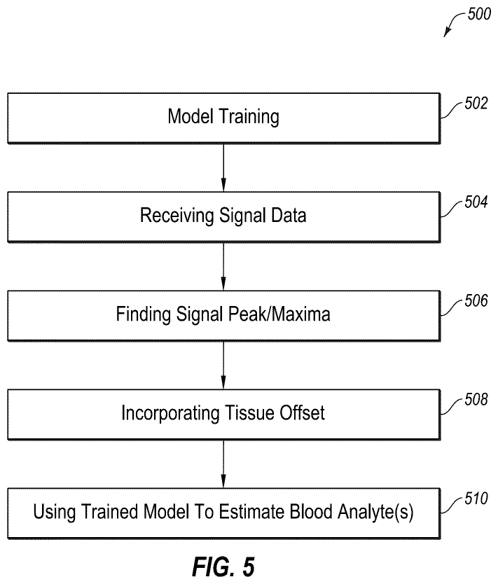

FIG. 5 is a flowchart illustrating a more generalized method 500 for estimating and/or predicting blood analyte conditions, such as blood analyte concentrations, according to some implementations. Method 500 begins at 502, at which point a model is trained. This training may be done using any of the steps and/or techniques described above. In some cases, this training may be done using Beer-Lambert inversion training algorithms and/or methods. In some cases, the training may involve use of certain features, such as the JMLS feature described herein. The training may comprise use of a design matrix, least-squares techniques, singular value decomposition, and/or nonlinear optimization, which may include gradient-based methods.

Following training, method 500 may comprise receiving signal data at 504. Again, such data is preferably non-invasively received from a spectrometer or other detector/sensor. In some cases, the signal data may be multi-channel signal data, which may comprise a plurality of wavelengths, in some cases within one or more of the wavelength ranges disclosed above. As also mentioned above, the signal data may in some cases be received from a blood monitoring device and/or system, which may comprise a wearable device, such as a wrist monitor, that may be configured with the aforementioned emitter(s) and sensor(s).

A peak, maxima, and/or maximum of the signal received may then be identified within a predetermined time period and/or range at step 506. For example, in some implementations, a peak of the signal may be identified within a single pulse of a pulsatile signal, such as a single beat of a heartbeat signal.

Similarly, step 508 may comprise finding a valley, minima, and/or minimum of the signal within the predetermined time period and/or range. Again, in implementations in which the predetermined time period and/or range comprises a single pulse and/or heartbeat, step 508 may comprise finding a valley of the signal within a single beat of a heartbeat signal.

In some implementations, tissue offset may optionally be incorporated into the model at step 510. In some cases, this offset may be applied by adding offset parameters, such as multiplicative offset parameters in some such cases, to a nonlinear optimization algorithm.

The processed data may then be applied to the trained model, such as a Linear or Nonlinear Beer-Lambert inversion model, to provide an estimate of one or more blood analytes at step 512. In some cases, step 512 may comprise estimating and/or predicting a concentration of a blood analyte, such as blood glucose, for example, and/or any of the other analytes previously mentioned.

As with any of the methods described herein, one or more of the steps of method 500 may be omitted in certain implementations. For example, the training step may be omitted and step 512 may be performed on a previously trained model. Similarly, the application of tissue offset, while potentially useful in certain applications, may be omitted in various contemplated implementations.

In certain embodiments, a particular software module may comprise disparate instructions stored in various locations of a memory device, which together implement the described functionality of the module. Indeed, a module may comprise a single instruction or many instructions, and may be distributed over several different code segments, among different programs, and across several memory devices. Some embodiments may be practiced in a distributed computing environment where tasks are performed by a remote processing device linked through a communications network. In a distributed computing environment, software modules may be located in local and/or remote memory storage devices.

In addition, data being tied or rendered together in a database record may be resident in the same memory device, or across several memory devices, and may be linked together in fields of a record in a database across a network. Furthermore, embodiments and implementations of the inventions disclosed herein may include various steps, which may be embodied in machine-executable instructions to be executed by a general-purpose or special-purpose computer (or another electronic device). Alternatively, the steps may be performed by hardware components that include specific logic for performing the steps, or by a combination of hardware, software, and/or firmware.

Embodiments and/or implementations may also be provided as a computer program product including a machine-readable storage medium having stored instructions thereon that may be used to program a computer (or other electronic device) to perform processes described herein. The machine-readable storage medium may include, but is not limited to: hard drives, floppy diskettes, optical disks, CD-ROMs, DVD-ROMs, ROMs, RAMs, EPROMs, EEPROMs, magnetic or optical cards, solid-state memory devices, or other types of medium/machine-readable medium suitable for storing electronic instructions. Memory and/or datastores may also be provided, which may comprise, in some cases, non-transitory machine-readable storage media containing executable program instructions configured for execution by a processor, controller/control unit, or the like.

It will be understood by those having ordinary skill in the art that changes may be made to the details of the above-described embodiments without departing from the underlying principles presented herein. Any suitable combination of various embodiments, or the features thereof, is contemplated.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Throughout this specification, any reference to “one embodiment,” “an embodiment,” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, inventive aspects lie in a combination of fewer than all features of any single foregoing disclosed embodiment. It will be apparent to those having skill in the art that changes may be made to the details of the above-described embodiments without departing from the underlying principles set forth herein.

Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, a required, or an essential feature or element. The scope of the present invention should, therefore, be determined only by the following claims.

1. A method for estimating blood analytes non-invasively, the method comprising the steps of:
training a model of a relationship between a blood analyte and a non-invasive signal, wherein the model uses a feature comprising at least two distinct electromagnetic radiation wavelengths, and wherein the model comprises a Beer-Lambert inversion model;
receiving signal data from a non-invasive blood monitor using the at least two distinct electromagnetic radiation wavelengths; and
using the trained model to estimate a blood analyte condition associated with the blood analyte.

2. The method of claim 1, wherein the signal comprises a pulsatile signal.

3. The method of claim 2, wherein the pulsatile signal comprises a heartbeat.

4. The method of claim 3, wherein the feature comprises use of signal values taken at a time of systole and a time of diastole of the heartbeat for two independent wavelengths of the at least two distinct electromagnetic radiation wavelengths.

5. The method of claim 4, wherein the signal values comprise a maximum value and a minimum value taken during a single heartbeat.

6. The method of claim 1, wherein the blood analyte condition comprises a concentration of the blood analyte.

7. The method of claim 1, wherein the blood analyte comprises glucose.

8. The method of claim 1, wherein the blood analyte comprises oxygen.

9. The method of claim 1, wherein the model is configured to estimate a tissue-dependent DC offset component of the signal data.

10. The method of claim 9, further comprising processing the signal data to extract the tissue-dependent DC offset component.

11. The method of claim 10, further comprising evaluating the processed signal data to provide an estimate of a blood analyte condition using only a frequency component corresponding to a heart rate pulse of a user of the non-invasive blood monitor and a non-pulsatile blood component corresponding to light reflected from within a blood vessel from the signal data.

12. The method of claim 1, wherein the Beer-Lambert model comprises a linear model.

13. The method of claim 1, wherein the Beer-Lambert model comprises a non-linear model.

14. A method for estimating a blood analyte concentration using data from a non-invasive blood monitor, the method comprising the steps of:
receiving signal data from a non-invasive blood monitor, wherein the signal data includes data associated with at least two distinct electromagnetic frequencies; and
using a trained model to estimate a blood analyte concentration, wherein the trained model comprises a Beer-Lambert inversion model.

15. The method of claim 14, wherein the trained model uses a feature using an equation comprising maximum and minimum values of a signal at two distinct electromagnetic frequencies.

16. The method of claim 15, wherein the equation comprises values of the signal at a time of systole and a time of diastole of a heartbeat for the two distinct electromagnetic frequencies.

17. The method of claim 16, wherein the maximum value corresponds to the time of diastole and the minimum value corresponds to the time of systole.

18. The method of claim 14, wherein the signal data comprises:
a frequency component corresponding to a heart rate pulse of a user of the non-invasive blood monitor;
a non-pulsatile blood component corresponding to light reflected from within a blood vessel; and
a tissue-dependent DC offset component.

19. The method of claim 18, further comprising extracting the tissue-dependent DC offset component from the signal data.

20. The method of claim 19, wherein the step of using a trained model to estimate a blood analyte concentration comprises estimating the blood analyte concentration using only the frequency component and the non-pulsatile blood component.
